(19) United States
(12) Patent Application Publication
(10) Pub. No.: US 2005/0251203 A1
(43) Pub. Date: Nov. 10, 2005

(54) Ferrule

(76) Inventor: Oleg Shikhman, Trumbull, CT (US)

Correspondence Address: CANTOR COLBURN, LLP, 55 GRIFFIN ROAD SOUTH, BLOOMFIELD, CT, 06002, US

(21) Appl. No.: 10/988,996

(22) Filed: Nov. 15, 2004

Related U.S. Application Data
(60) Provisional application No. 60/520,236, filed on Nov. 14, 2003.

(51) Int. Cl. A61B 17/08

(52) U.S. Cl. 606/213

(57) ABSTRACT

An exemplary improved ferrule includes a biocompatible body portion, the body portion having an aperture therethrough, the body portion also having a first flared region and, optionally, a biocompatible cover material provided on at least a portion of the biocompatible body portion.

### CROSS REFERENCE TO RELATED APPLICATIONS

The present application claims priority to U.S. Provisional Patent Application No. 60/520,236 filed Nov. 14, 2003, the entire contents of which are incorporated herein by reference in its entirety.

### BACKGROUND

The present disclosure relates to an instrument and a method for closing a hole or puncture in a blood vessel. More particularly, this disclosure relates to an improved ferrule closure for a hole or puncture in a blood vessel.

When performing catheterization procedures, such an angiography or angioplasty, a catheter is generally introduced into the vascular system by first penetrating the skin, underlying muscle tissue and blood vessel with a sharpened hollow needle. Next, a guide wire is commonly inserted through the lumen of the hollow needle and is caused to enter the selected blood vessel. Subsequently, the needle is typically slid off the guide wire and a combination of a dilator and an introducer (or an introducer alone) are fed over the guide wire and pushed through the skin to enter the vessel. The guide wire can then be removed and the desired catheter to carry out the procedure is fed through the lumen of the introducer and advanced through the vascular system until the working end of the catheter is appropriately positioned. Following the conclusion of the catheterization procedure, the working catheter will be withdrawn and, subsequently, the dilator and/or introducer will also be removed from the wound.

At this point in the procedure, the vessel leakage must be controlled in order to stem the flow of blood through the puncture. Because it is common practice to administer a blood thinning agent to the patient prior to many of the catheterization procedures, stemming the blood flow can be troublesome. A common method of sealing the wound is to maintain external pressure over the vessel until the puncture naturally seals. This method of puncture closure typically takes about thirty minutes, with the length of time usually being greater if the patient is hypertensive or anti-coagulated. In some anti-coagulated patients, the introducer is left in place for hours to allow the anti-coagulant to wear off. When human hand pressure is utilized, it can be uncomfortable for the patient and can use costly professional time on the part of the hospital staff. Other pressure techniques, such as pressure bandages, sandbags or clamps, have been employed, but these devices also require the patient to remain motionless for an extended period of time and the patient must be closely monitored to ensure their effectiveness.

Suture securing instruments are being developed to assist in finalizing the suturing procedure. The presently described ferrule finds application in such suture securing instruments, among others.

### SUMMARY

An exemplary improved ferrule includes a biocompatible body portion, the body portion having an aperture therethrough, the body portion also having a first flared region. Another exemplary ferrule includes a biocompatible cover material provided on at least a portion of the biocompatible body portion. In one embodiment, the biocompatible body portion is a stainless steel material, a titanium material or the like. In another embodiment, the biocompatible cover material is an expanded polytetrafluoroethylene material or the like. In another embodiment, the biocompatible cover material is a compliant material. In another embodiment, the biocompatible body portion is in a cylindrical form, having an aperture therethrough. In another embodiment, the aperture is configured to receive at least on suture strand, and the biocompatible body portion is deformable to secure the suture strand within the aperture. In another embodiment, the biocompatible cover material is provided at least partially within the aperture of the biocompatible body portion. In another embodiment, the biocompatible body portion has a first end portion and a second end portion, and the second end portion is flared outwardly from the cylindrical body portion. In another embodiment, the biocompatible cover material is provided at least partially on the second end portion. In another embodiment, the biocompatible cover material surrounds the outward flare of the cylindrical body portion.

The above-discussed and other features and advantages of the present invention will be appreciated and understood by those skilled in the art from the following detailed description and drawings.

### BRIEF DESCRIPTION OF THE DRAWINGS

Referring to the FIGURES wherein like elements are numbered alike in the several FIGURES:

FIG. 1 shows an cross sectional view of a first exemplary embodiment;

FIG. 2 shows a cross sectional view of a second exemplary embodiment;

FIG. 3 shows a cross sectional view of an exemplary embodiment positioned at the tip of a suture securing device; and

FIG. 4 shows a cross sectional view of another exemplary embodiment positioned at the tip of a suture securing device.

### DETAILED DESCRIPTION OF THE INVENTION

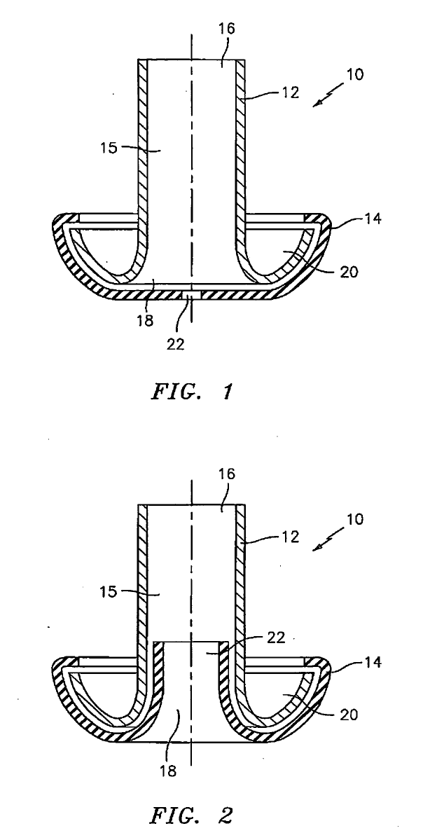

Referring to FIG. 1, an exemplary ferrule 10 is illustrated, comprising a biocompatible body portion 12 and a biocompatible cover portion 14 provided on a portion thereof. An aperture 15 is provided through a portion of the body portion 12. In the illustrated exemplary embodiment, the body portion 12 has a first end portion 16 and a second end portion 18, and the second end portion 18 includes a flared region 20 relative to the first end portion 16. The flared region 20 may be included where, e.g., it is desirable for facile loading of suture or other material therethrough or where it is desirable to facilitate movement of the ferrule through some medium (e.g., down a tissue tract). The flared region 20 may also be provided where it is desirable for the second end portion 18 to have a larger cross sectional width than the first end portion 16. The term “flared” should be construed to include any portion that extends outwardly from a portion of the body portion, whether it be the first end portion 16, part of the second end portion 18 or any region in-between. Furthermore, the flared region 20 may extend outwardly at any angle, including a ninety degree angle, from the remainder of the body portion 12.

Referring still to FIG. 1, the illustrated exemplary embodiment includes cover material 14 provided on the second end portion 18. More specifically, the illustrated exemplary embodiment illustrates the cover material 14 as encircling the flared region 20. Also as illustrated by the exemplary embodiment, the cover material may include a slit or other aperture 22 near the aperture 15 of the body portion 12.

Referring now to FIG. 2, a second exemplary embodiment is illustrated. In this exemplary embodiment, the cover material 14 is provided at least partially within the aperture 15 of the body portion. As in the above described exemplary embodiment, the cover material 14 includes an aperture 22.

The biocompatible body portion may be non-bioabsorbable or bioabsorbable. In one embodiment, the body portion is non-bioabsorbable, such as a stainless steel material, a titanium material or the like. In another embodiment, the body portion is bioabsorbable, e.g., manufactured from a bioabsorbable polymer, such as a homopolymer, copolymer or a blend obtained from one or more monomers selected from the group consisting of glycolic acid, lactide, lactic acid, p-dioxane, E-caprolactone and trimethylene carbonate.

The biocompatible cover material may also be non-bioabsorbable or bioabsorbable. In one exemplary embodiment, the biocompatible compliant material is an expanded polytetrafluoroethylene material, a Teflon material, or the like. In another embodiment, the biocompatible cover material is compliant, such that it will at least conform around adjacent material (e.g., around suture secured within the body portion. The cover material may be secured to the body portion by any convenient mechanism, e.g. by bonding, gluing, shrinking, forming around the body, by elastic properties of the cover material, etc.

In another embodiment, the aperture of the body portion is configured to receive at least one suture strand, and at least a portion of the body portion is deformable to secure the suture strand within the aperture. In such embodiment, the body portion may be deformed by any convenient device or method, e.g. as that described by U.S. Pat. No. 5,643,289 to Sauer, the contents of which are specifically incorporated herein by reference, or co-pending U.S. patent application Ser. No. 10/037,899, filed Oct. 22, 2001, the entire contents of which are specifically incorporated herein by reference.

Referring now to the exemplary embodiment illustrated at FIG. 3, an exemplary ferrule 10 is illustrated as loaded onto a positioning and/or crimping device 30. Covering material 14 is provided along portions of the flared region 20. As illustrated, covering material 14 also extends onto a first end portion 32 of the positioning and/or crimping device 30, for example, to facilitate insertion of the device 30 into position in the wound of a patient.

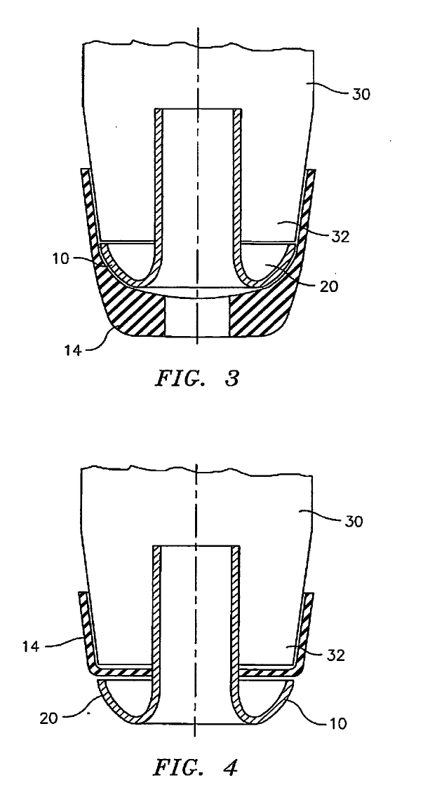

Referring now the exemplary embodiment illustrated at FIG. 4, an exemplary ferrule 10 is illustrated as loaded onto a positioning and/or crimping device 30. Covering material 14 is provided adjacent (on the backside) of portions of the flared region 20. As illustrated, covering material 14 also extends onto a first end portion 32 of the positioning and/or crimping device 30, for example, to facilitate insertion of the device 30 into position in the wound of a patient. In use, the covering material 14 may remain on the device 30 after securing of the ferrule 10 at the wound. Alternately, the covering material 14 may remain with the ferrule 10 and serve (as with the rest of the described embodiments having covering material 14) as a covering material for the portions of the flared region 20 or generally as a pledget for the wound after securing the ferrule 10 at the wound.

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A biocompatible ferrule, comprising:
a biocompatible body portion having a first flared region.

2. The biocompatible ferrule of claim 1, wherein said body portion has a first end portion and a second end portion, and wherein the first flared region is provided on the first end portion.

3. The biocompatible ferrule of claim 2, wherein the first flared region extends gradually outwardly from the longitudinal axis of the body portion at increasing angles.

4. The biocompatible ferrule of claim 3, wherein the first flared region extends up to about 45 degrees from the longitudinal axis of the body portion.

5. The biocompatible ferrule of claim 4, wherein the first flared region extends up to about 90 degrees from the longitudinal axis of the body portion.

6. The biocompatible ferrule of claim 5, wherein the first flared region extends past 90 degrees such that it changes direction with regard to the longitudinal axis of the body portion.

7. The biocompatible ferrule of claim 5, wherein the first flared region extends past 90 degrees such that it changes direction with regard to the longitudinal axis of the body portion, and wherein the flared region continues to gradually extend outwardly up to about 140 degrees.

8. The biocompatible ferrule of claim 2, wherein such flare is measured solely with reference to the outer surface of the body portion.

9. The biocompatible ferrule of claim 2, wherein such flare additionally expands the inner diameter of the body portion where such flare occurs.

10. The biocompatible ferrule of claim 2, further comprising a cover material provided on or adjacent to at least a portion of said first flared region.

11. The biocompatible ferrule of claim 10, wherein said cover material completely covers said first flared region.

12. The biocompatible ferrule of claim 10, wherein said cover material includes an aperture near an aperture of the body portion.

13. The biocompatible ferrule of claim 10, wherein said cover material is provided at least partially within an aperture of the body portion.

14. The biocompatible ferrule of claim 10, wherein said cover material is non-bioabsorbable.

15. The biocompatible ferrule of claim 14, wherein said cover material is stainless steel or titanium.

16. The biocompatible ferrule of claim 10, wherein said cover material is bioabsorbable.

17. The biocompatible ferrule of claim 16, wherein said cover material is a homopolymer, copolymer or a blend obtained from one or more monomers selected from the group consisting of glycolic acid, lactide, lactic acid, p-dioxane, E-caprolactone and trimethylene carbonate.

18. The biocompatible ferrule of claim 10, wherein said cover material is an expanded polytetrafluoroethylene material or a Teflon material.

19. The biocompatible ferrule of claim 10, wherein said cover material is compliant, such that it will at least conform around adjacent material

20. The biocompatible ferrule of claim 10, wherein said cover material is secured to the body portion by bonding, gluing, shrinking, forming around the body or by elastic properties of the cover material.

21. The biocompatible ferrule of claim 2, wherein an aperture of the body portion is configured to receive at least one suture strand, and at least a portion of the body portion is deformable to secure the suture strand within the aperture.

22. A biocompatible ferrule positioning or crimping device, comprising:
a positioning or crimping device body having a first end portion, the first end portion configured to receive a biocompatible ferrule; and
a biocompatible ferrule configured to be received in said first end portion, the biocompatible body portion having a first flared region.

23. The device of claim 22, further comprising a cover material provided on or adjacent to at least a portion of said first flared region.

24. The device of claim 23, wherein said cover material is provided on or adjacent to at least a portion of said first end portion.
